(19) United States
(12) Patent Application Publication
(10) Pub. No.: US 2004/0167763 A1
(43) Pub. Date: Aug. 26, 2004

(54) Information processing method for evaluating biochemical pathway models using clinical data

(76) Inventor: Michael N Liebman, Paoli, PA (US)

Correspondence Address: FOLEY AND LARDNER, SUITE 500, 3000 K STREET NW, WASHINGTON, DC, 20007, US

(21) Appl. No.: 10/478,313

(22) PCT Filed: May 31, 2002
(86) PCT No.: PCT/US02/17031

(51) Int. Cl. G06G 7/48; G06G 7/58; G06F 19/00; G01N 33/48; G01N 33/50

(52) U.S. Cl. 703/11; 702/20

(57) ABSTRACT

A method of evaluating a biochemical pathways models using clinical data, includes representing the biological model within a computing system, in the form of a hierarchy of Petri nets or stochastic activity nets, wherein the nodes of the net represent biological or biochemical components, and the arcs correspond to the flow of biochemical components in the model. A time series of measurements of some of the biochemical components described by the model are recorded and an observed pattern of relationships between inputs and outputs of the respective nodes is compared to an expected pattern of the relationships to determine whether the model describes the behavior of the biochemical components.

### CROSS-REFERENCE TO RELATED PATENT APPLICATIONS

The present application claims the benefit of priority under 35 U.S.C. § 119(e) of provisional application No. 60/294,639 filed on Jun. 1, 2001. The content of this provisional application is incorporated by reference herein in its entirety.

### BACKGROUND OF THE INVENTION

In order to analyze and model a biological system and simulate its behavior in normal function and in dysfunction, it is necessary to integrate a wide range of data and observations. The conventional development of a biological pathway model requires the measurement of component enzyme reaction kinetics in vitro, and the generation and solution of the composite set of differential equations. This approach can be limited by the experimental difficulties in the isolation and purification of component enzymes, and by difficulties in replicating in vivo reaction conditions. These experimental difficulties can lead to measurements that inaccurately reflect the in vivo activity of the enzymes.

Furthermore, for some biological pathways, the input components are not fully characterized or cannot be easily measured. In some systems for which one is interested in developing models, there is only limited data available on the components of the pathway itself.

Petri nets and stochastic activity nets (SANs) have been used as generalized systems models, and have been applied to the modeling of biochemical pathways.

### SUMMARY OF THE INVENTION

The present invention provides an improved ability to model biochemical pathways by means of a Petri net or stochastic activity net, and thereby to avoid the uncertainties that arise in determining rate constants for a pathway model based on ordinary differential equations. Additionally, this invention obviates the need to measure all input components of a model in order to utilize it, by employing a search strategy to reconstruct unknown inputs based on measured outputs.

The present invention provides a model of a biological system, in the form of a hierarchy of Petri nets or Stochastic Activity Nets. While the Petri nets and Stochastic Activity Nets are the preferred models, one skilled in the art would recognize that the principles of the present invention may be used with other similar models all of which are considered within the scope of the present invention. The top levels of the hierarchy may represent an entire organism or organ system. Lower levels may represent tissues and cells, and the lowest levels may represent individual biochemical components of metabolic pathways. The components and outputs of each node in the network are labeled. Input labels are created by inspecting the network components, to determine whether an increase in token flow at the input will cause an increase or decrease in token flow at each output of the node. The network model can be compared to observed data from living organisms, to determine the consistency of the model, and in order to elucidate the roles of the various components of the biological system under study.

According to one aspect of the present invention, a method for evaluating a biological model, includes the steps of: (a) representing the biological model within a computing system, in the form of a hierarchy of Petri nets or stochastic activity nets, wherein the nodes of the net represent biological or biochemical components, and the arcs correspond to the flow of biochemical components in the model; (b) recording a time series of measurements of some of the biochemical components described by the model, wherein these measurements are made on samples from one or more humans or other living organisms; (c) entering and storing those observations as a data set in the computing system, which performs the subsequent steps as automated computations; (d) optionally selecting for subsequent analysis a subset of the data set, based on patient demographic information or history of prior treatment; (e) for each time series, calculating the rate of change of some or all variables with respect to time, and augmenting the data set with that information; (f) for each input to a network node, labeling the input with the expected effect of an increase in token flow at the input on each output of the node, either “increasing” or “decreasing”; (g) comparing in a qualitative or quantitative sense the pattern of increases and decreases of measured biochemical levels versus the pattern of increases and decreases in flow rates of tokens through the corresponding nodes; and (h) marking the biochemical variables and measurements for which the expected effect is an increase while the biochemical measurements show a decrease, or vice versa, the marking indicating that the model does not adequately describe the behavior of those biochemical components.

In another aspect of the present invention, for models which are consistent in that they have no markings from step (h) above, further marking nodes Whose outputs are connected, directly or indirectly, to inputs of a relatively large number of nodes compared to the average connectivity of the network, the marking consisting of an annotation that the node is a potential key regulatory factor.

In another aspect, the method of the present invention relates to a method of evaluating a biological model, including the steps of: (a) representing the biological model within a computing system, in the form of a hierarchy of Petri nets or stochastic activity nets, wherein the nodes of the net represent biological or biochemical components, and the arcs correspond to the flow of biochemical components in the model; (b) recording a set of measurements of some of the biochemical components described by the model, wherein these measurements, are made on samples from one or more humans or other living organisms, wherein the set of patients is divided into subsets representing patients with a disease, various diseases, and/or healthy patients; (c) entering and storing those observations as a data set in the digital computer system, which performs the subsequent steps as automated computations; (d) optionally selecting for subsequent analysis a subset of the data set, based on patient demographic information or history of prior treatment; (e) for subset of the data, calculating the change of some or all measured variables with respect to the corresponding value of that variable for healthy patients or diseased patients in a different subset, and augmenting the data set with that information; (f) for each input to a network node, labeling the input with the expected effect of an increase in token flow at the input on each output of the node, either “increasing” or “decreasing”; (g) comparing in a qualitative or quantitative sense the pattern of increases and decreases of measured biochemical levels in various disease states versus the pattern of increases and decreases in flow rates of tokens through the corresponding nodes; (h) marking the biochemical variables and measurements for which the expected effect is an increase while the biochemical measurements show a decrease, or vice versa, the marking consisting of an annotation that the model does not adequately describe the behavior of those biochemical components; and (i) for models which are consistent in that they have no markings from step (h) above, further marking nodes whose outputs are connected, directly or indirectly, to inputs of a relatively large number of nodes compared to the average connectivity of the network, the marking consisting of an annotation that the node is a potential key regulatory factor.

In one aspect, the measurements in step (b) may be taken from diseased patients, and the levels of the biochemical components identified in step (i) may be subjected to cluster analysis as a means of stratifying a disease.

In another aspect, the set of patients includes both diseased and non-diseased individuals, and biochemical components are identified in step (i) for the purpose of developing a biochemical diagnostic test for the disease under study. Alternatively, the set of patients can include individuals at various stages of disease, and the biochemical components are identified in step (i) for the purpose of developing a method to measure disease progression or stage.

In a further aspect of the present invention, the biochemical components identified in step (i) are subsequently utilized for the purpose of developing a predictive test for the clinical course of the disease under study, or for determining the best treatment for an individual patient. Alternatively, the biochemical components identified in step (i) or their associated enzymes are subsequently employed as targets for the development of therapeutic drugs.

### BRIEF DESCRIPTION OF THE DRAWINGS

FIG. 1 is diagram showing an exemplary Petri net model.

FIG. 2 is an exemplary biological system modeled using a Stochastic Activity Net.

FIG. 3 is diagram illustrating the labeling of a model according to the present invention.

FIG. 4 is a diagram illustrating a SAN model for evaluating normal blood coagulation.

FIGS. 5(a)-(e) are five simplified SAN models derived from the SAN model of FIG. 4.

FIG. 6 is a table showing the common places necessary to complete the composed model of coagulation.

FIG. 7(a) shows the experimental data for the activation reactions of the five factors used in the SAN model.

FIG. 7(b) shows the response of the initial model of these five factors against simulation time.

FIG. 7(c) illustrates a corresponding factor X activation curve.

FIGS. 8(a)-(c) illustrate the final model achieved iteratively.

FIGS. 9(a)-(c) show three graphs illustrating hemophilia simulations.

FIG. 10 shows the result of another study with factor IX defect hemophilia.

FIG. 11(a)-(c) illustrate three simulations to illustrate sensitivity analysis of coagulation factors.

### DETAILED DESCRIPTION OF THE PREFERRED EMBODIMENTS

Petri nets are mathematical modeling tools that were first introduced in 1962. They allow an investigator to model a dynamic process in terms of discrete events that can be monitored during a process simulation, to evaluate the flow of material through a system. Petri nets have been used to model information flow in computer networks, as well as manufacturing processes and other processes involving the flow of physical material.

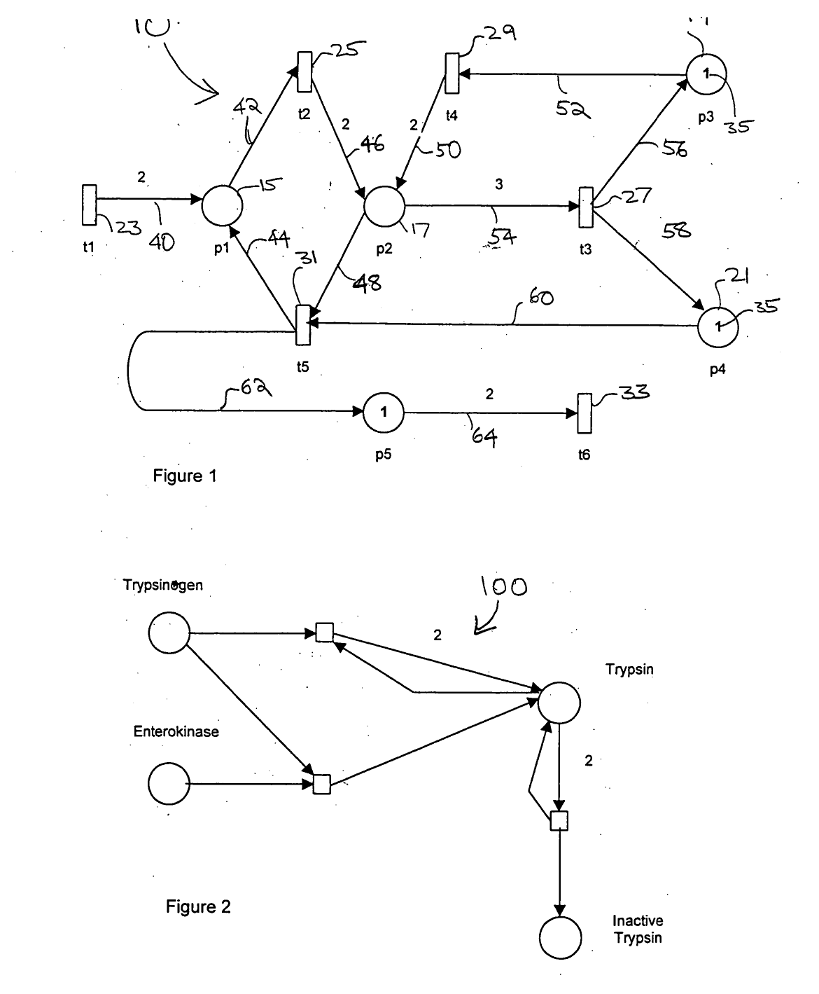

Petri nets are composed of “nodes,” which include both “places” and “transitions,” “directed arcs,” which connect places and transitions, and “tokens,” which represent discrete quantities of material at a given place. Places are used to collect tokens, the elements used to represent material flowing through the system. Transitions move tokens between places. A Petri net system 10 is depicted in FIG. 1. The places (15, 17, 19, 21) are represented by open circles and are labeled p1-p5. The transitions (23-33) are represented by rectangles and are labeled t1-t6. Tokens 35 are represented as numbers within places. The set of tokens in each place at the start of simulation is called the “initial marking,” M₀, of the Petri net. Finally, the places and transitions are connected together by means of directed arcs (40-64). Each of the arcs dictates token flow, i.e., the change in token marking, with respect to direction and associated rate of transfer. The numbers associated with specific arcs represent their weight, such that if an arc has a weight of two, then two tokens are moved between nodes at a time. Arcs without designated weights have the default arc weight of one.

Token flow occurs between places, dictated by whether the corresponding transition is appropriately enabled. If the number of tokens in each of the input places is greater than or equal to the associated arc weights connecting the input places to the transition, then that transition is enabled to transfer tokens. In FIG. 1, transition t4 (29) is enabled because the number of tokens indicated in its corresponding input place, p3 (19), is equal to the arc weight connecting the input place to the transition. Thus, transition t4 (29) is enabled to take one token from place p3 (19) and put two tokens in place p2 (17). Transition t5 (35) is not enabled, because, even though place p4 (35) satisfies its input requirements, place p2 (17) does not.

Special instances exist where a transition is connected only to an input place or an output place. These transitions are termed “sink transitions” and “source transitions,” respectively, and act to continuously remove tokens from (sink), or supply tokens to (source) the network. In FIG. 1, transition t1 (23) functions as a source transition, and transition t6 (33) functions as a sink transition.

Transition firing or execution in Petri nets can be performed by two distinct methods, sequential and parallel execution. Sequential execution is performed by executing only one enabled transition during each cycle through the model, where the choice of the transition to be executed is made randomly. Parallel execution occurs by firing each enabled transition during each cycle through the model. Either choice is applicable to the present invention.

Petri nets themselves are limited in their capabilities for biological simulation, in that there are few parameters that can be adjusted to control the flow of tokens through the system.

Stochastic Activity Nets (SANs) are an extension of Petri nets that allow the incorporation of probabilistic functions to control transitions. In the preferred embodiment of this invention, Stochastic Activity Nets are used to perform the simulation. Software such as UltraSAN can be utilized to perform biological simulations with Stochastic Activity Nets.

The UltraSAN environment consists of five process elements: places, activities, gates, arcs, and tokens. Places and tokens of a SAN correspond directly to those same elements in a Petri net. Activities are an extension of the Petri net transition, and can either be designated as instantaneous or timed. Furthermore, activities have the added feature of allowing case probabilities that can alter an activity's response under varying input conditions. Gates and arcs replace the directed arcs of Petri nets. Gates dictate token flow between places and activities. A gate must be used in conjunction with arcs connecting it to the associated place and activity, unless the token transfer between nodes is one, in which case only a single arc is required.

The activity process element differs from its analogous Petri net transition in how it responds within the network. Simulation is done sequentially, but is based on simulation time, a computer-generated time scale. Each timed activity in a SAN contains a user-defined “Activity Time Distribution Function” that dictates the amount of time it takes for tokens to pass from the input places to the output places. This generates more exacting control in the modeling process using SANs.

Another property of the activity process element is its ability to respond separately to distinct states of the network. For example, given that the number of tokens in an input place will be a value of zero, one, or two, a timed activity can be defined to give one of two different outputs, and can be assigned a probability for each occurrence.

UltraSAN allows the developer to decompose a large, complex, SAN system into simpler systems, such that these subsystems can be modeled and refined in parallel, and then integrated into a composite model that can be further refined. This hierarchical approach to modeling is used in the one embodiment of this invention. As an illustration, a discussion of a qualitative modeling of normal blood coagulation and its pathological states using a SAN is discussed at the end herein.

Upon completion of the development of a model, simulations can be performed to evaluate its response in comparison to predetermined inputs. During these simulations, software such as UltraSAN allows any individual place within the model to be viewed at any instant in time. Thus reactions can be simulated in terms of the token levels in various places as a function of time, where the places correspond to physical components of the biological model.

An example of a simple biological system 100 modeled by a SAN is shown in FIG. 2. The model represents trypsin activation and inactivation. The system involves three reactions. In the first reaction, enterokinase activates the zymogen, trypsinogen, when trypsinogen is presented to the duodenum from the pancreas. The membrane-bound enterokinase cleaves the N-terminal hexapeptide of trypsinogen to produce enzymatically active trypsin. Once trypsin is formed, it initially functions to catalyze the trypsinogen activation reaction itself, and as the concentration of trypsin increases, this reaction becomes predominant over the enterokinase activation reaction. Finally, as the concentration of active trypsin increases, an autolysis reaction is initiated, where trypsin acts on trypsin to inactivate it by endopeptidase cleavage. This reaction removes active trypsin from the system. This process functions as a feedback process wherein the trypsin level plateaus and is self-controlled from exceeding a definable level by initiating an autocatalytic inactivation process.

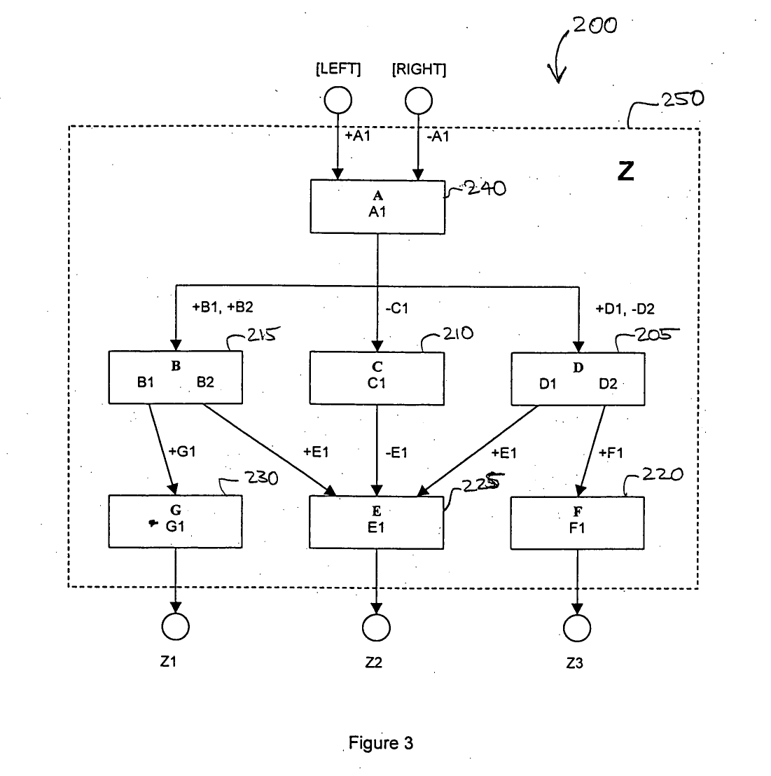

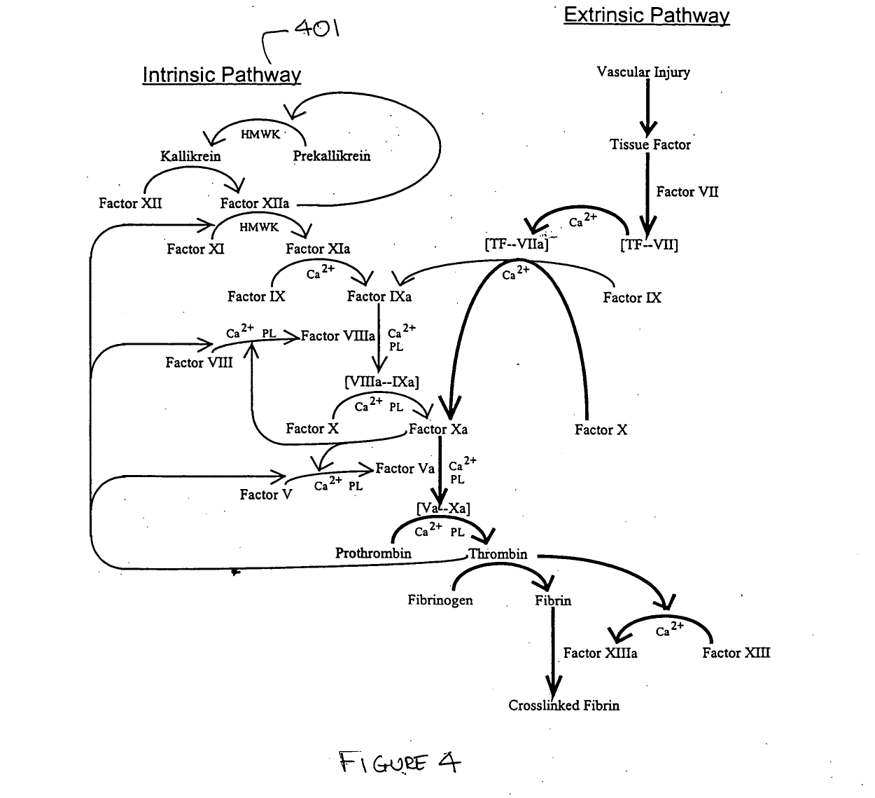

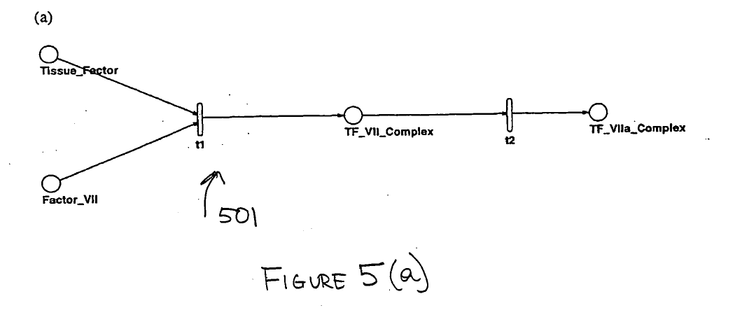

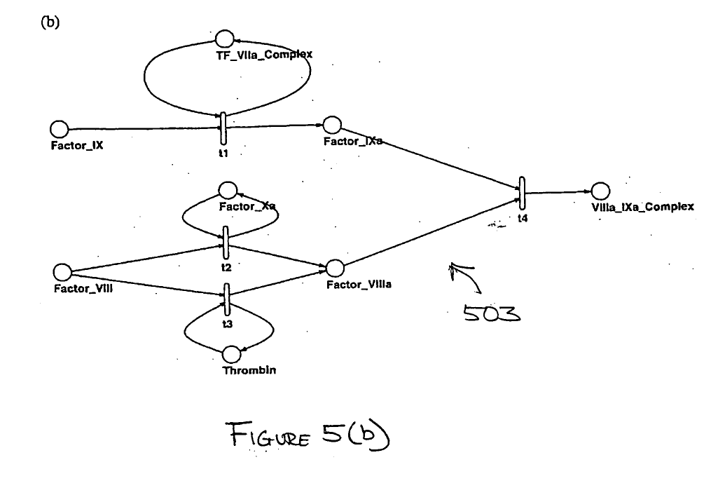

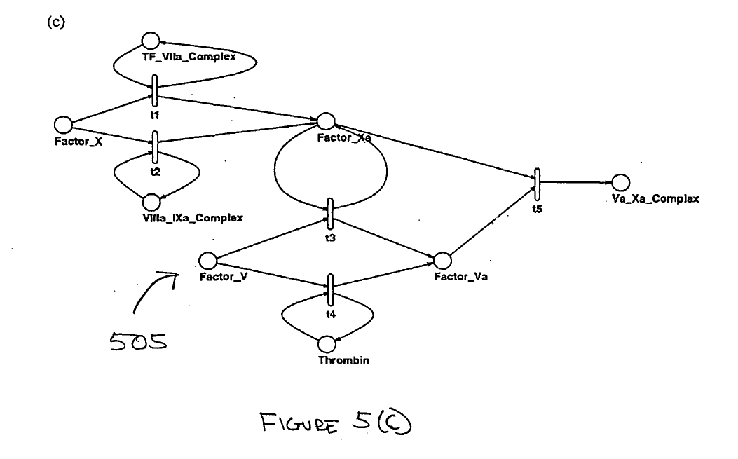

As shown in FIG. 3, a biological system 200 is described by a hierarchical set of Petri nets or Stochastic Activity Networks. At a given level of the hierarchy, each component is described as a labeled object with inputs and outputs. Each output is labeled with the place name and a number so that, for example, the two outputs of component D (205) are labeled D1 and D2. Inputs are labeled according to their effect on the outputs. For example, the label “+D1,−D2” indicates that an increase in token flow at this input will cause an increase in token flow at output D1, and a decrease in token flow at output D2.

The entire set of components A through G (205-240) can be viewed as a single component Z (250) at a higher level of the hierarchy. This component Z (250) then has two inputs (marked LEFT and RIGHT), and three outputs labeled Z1, Z2, and Z3. By examining the labels and topology of the network, it is clear that an increase in token flow at the LEFT input will cause an increase in token flow at outputs Z1 and Z2, and a decrease in token flow at output Z3. Thus the LEFT input is assigned a label of “+Z1 , +Z2, −Z3”. In a similar fashion, the RIGHT input is assigned a label of “−Z1, −Z2, +Z3”.

Once the model is built, all input nodes can be labeled as described above. Then the model can be compared against a set of data that contains biochemical or physiological measurements as a function of time. The rate of token flow through a place in the Petri/SAN model corresponds qualitatively to a measured quantity in the biological system under study.

The model and the data can be qualitatively or quantitatively compared for consistency. For example, if the biological quantity corresponding to the LEFT input of Z (250) is increasing over time, then the biological quantity corresponding to Z1 should be increasing over time as well. If this latter component is decreasing over time, then the data and the model are inconsistent.

Where inconsistency is found at a given level in the hierarchy, it tells the investigator to examine the model at a greater hierarchical level of detail, making additional data measurements as necessary, to identify and correct the source of the inconsistency in the model.

Once the model has been refined so that it is consistent with all available data, it is useful as a prescriptive and predictive description of the biological system under study. The investigator can determine the relative importance and roles of biological components by examining the topology of the network. Components with a high number of input and output connections are likely to be of great biological importance. Components closer to the root of tree-like structures in the network may be key regulatory factors. This information can be useful in the selection of target compounds for the development of therapeutic drugs, since it indicates the components that control the behavior of the modeled pathway as a whole. Finally, the investigator can identify components that regulate other components that are known to be of great importance.

If the important components identified in this manner are not modeled down to the most detailed level in the hierarchy, this may indicate to the investigator that it would be desirable to study these components in greater detail, in order to develop more fundamental models of their behavior.

Some of the uses of the model evaluation technique discussed herein include:

1. Evaluating the completeness of hypotheses concerning the topology and interactions of a biological pathway or process.

2. Development of a rational plan for experimental verification or identification of additional information as necessary to enhance a model's performance against observed data.

3. Evaluation of sensitivity of individual pathway components as to their ability to alter outputs produced by the pathway function.

4. Ability to evaluate the occurrence of single nucleotide polymorphisms or other sequence mutations in the pathway components as to their impact on the physiological response.

5. Ability to evaluate the change in protein expression levels as evidenced from proteomic or gene expression analysis of specific pathway components as to their impact on physiological response.

6 Prioritization of pathway components as targets for genotyping to identify polymorphisms in the population that may exhibit physiological differences among individuals.

7. Evaluation of individual pathway components as to their ability to alter pathway behavior upon intervention with therapeutic targeting.

8. Development of potential dosing models by evaluating the levels of alteration to a specific pathway component necessary to achieve a desired alteration in the physiological response.

9. Extension of all of the above to indicate simultaneous alterations to more than one component of the pathway.

The modeling, data storage, and computation steps discussed here may be accomplished using a computing system optionally connected to electronic network, such as a computer network. The computer network can be a public network, such as the Internet. The computing system typically includes a central processing unit (CPU) connected to a system memory. The system memory typically contains an operating system, a BIOS driver, and application programs. In addition, the computing system contains input devices such as a mouse or a keyboard, and output devices such as a printer and a display monitor, The computing system generally includes a communications interface , such as an Ethernet card, to communicate to the electronic network. Other computing systems may also connect to the electronic network which can be implemented as a Wide Area Network (WAN) or as an internetwork such as the Internet.

One skilled in the art would recognize that the above describes-a typical computer system connected to an electronic network. It should be appreciated that many other similar configurations are within the abilities of one skilled in the art and it is contemplated that all of these configurations could be used with the methods of the present invention. Furthermore, it should be appreciated that it is within the abilities of one skilled in the art to program and configure a computing system and related electronic signals and communications to implement the method steps of the present invention.

Furthermore, the present invention contemplates providing computer readable data storage media with program code recorded thereon -for implementing the method steps described in the present application.

Qualitative Modeling of Normal Blood Coagulation and its Pathological States Using Stochastic Activity Networks

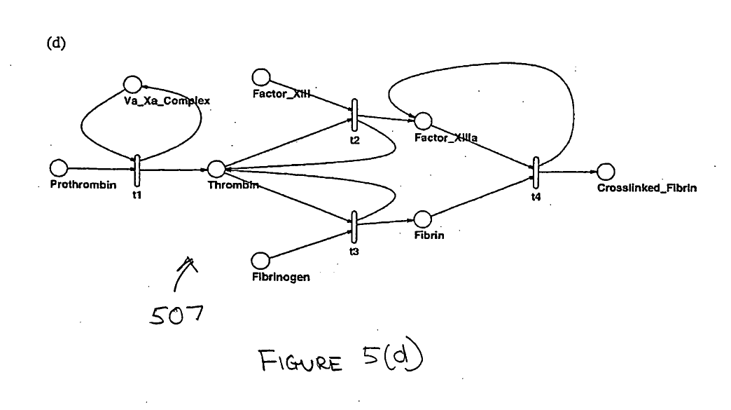

The basic procedure described above with respect to the trypsin system is used for modeling blood coagulation. The system to be modeled needs to first be described in terms of interactions and stoichiometry, and then experimental kinetic observations are required to refine the initial model to a final working model. The first step in applying this procedure required the delineation of the process from vascular injury through clot formation. Extrinsic blood coagulation is initiated by vascular injury which exposes tissue factor to the blood stream. This exposure results in activation of the extrinsic pathway which is shown as the bold set of reactions in FIG. 4. Once thrombin is produced by the initial cascade of zymogen activation reactions, it then participates in many feedback reactions, including activation of the intrinsic pathway which can supplement the extrinsic pathway. The intrinsic and extrinsic pathways 401 and 402 cooperate to produce crosslinked fibrin, i.e., the blood clot, from fibrin, thus effecting repair of the injury.

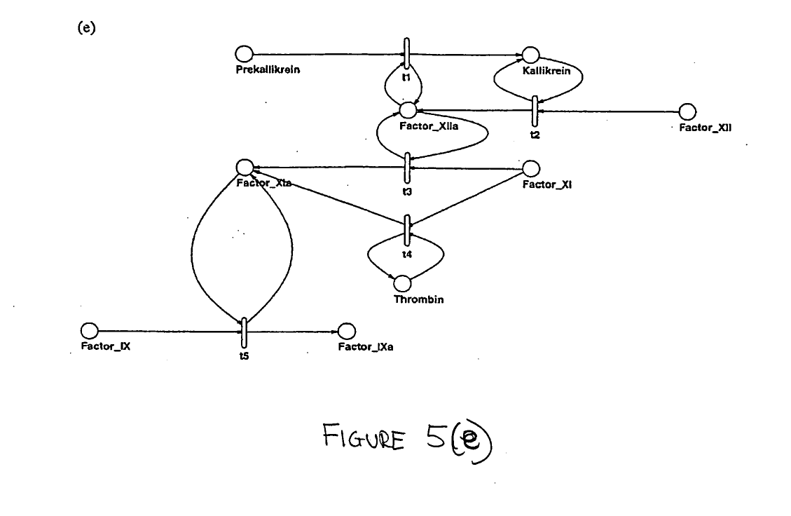

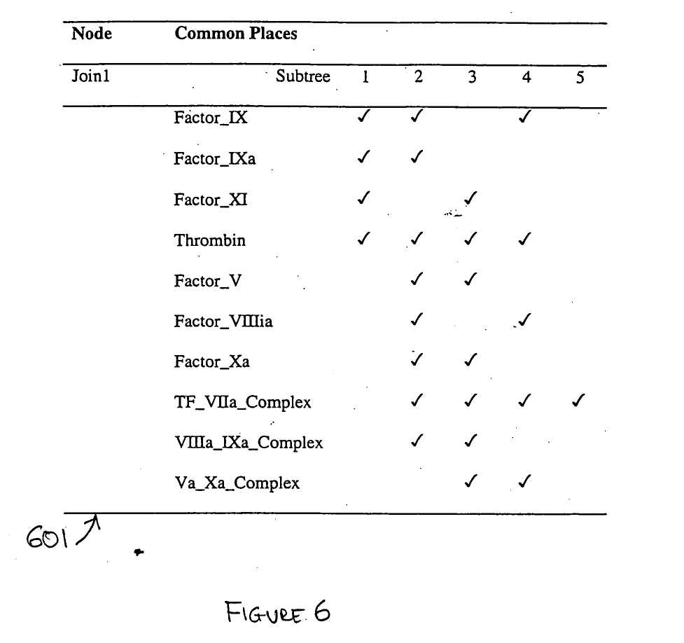

The initial SAN representation of the coagulation system is presented in FIG. 5. The figure shows five pathway segments which combine to yield the representation of the entire system. The segments include: 1) initiation 501 (FIG. 5(a)), 2) Factor VIII-Factor IX complex formation 503 (FIG. 5(b)), 3) Factor V-Factor X complex formation 505(Figure 5(c)), 4) clot formation 507 (FIG. 5(d)), and 5) the intrinsic pathway 509 (FIG. 5(e)). Each zymogen enzyme, as well as complex, are represented by a distinct place. These places are connected to activities by arcs, representing the reactions and stoichiometry of the coagulation system as it is presented in FIG. 4. For example, the representation presented in FIG. 5(a) shows that tissue factor and factor VII interact to form a 1:1 complex. Once the complex is formed, factor VII is activated to factor VIIa. This activated complex can then activate factor IX or factor X as is represented in FIG. 5(b) and FIG. 5(c), respectively. Thus, each of the reactions present in FIG. 4 has a corresponding schematic representation in one of the segments of FIGS. 5(a)-(e).

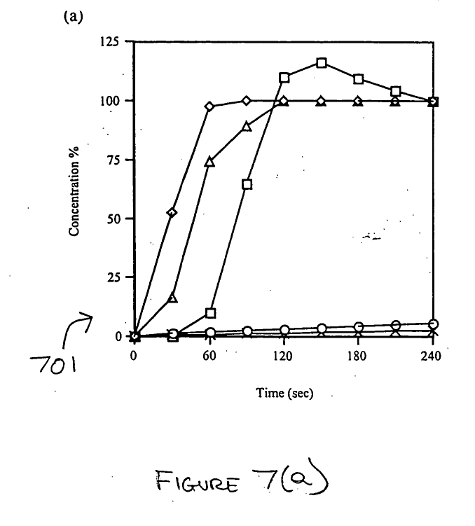

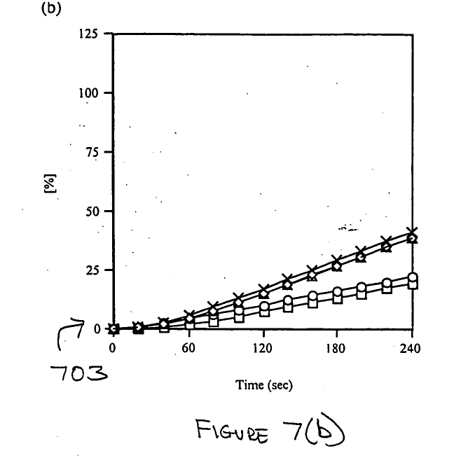

One of the key features of the UltraSAN tool is the potential to construct a hierarchical model from individual SANs. The model developed for coagulation provides an example of this process. Five separate SANs (FIGS. 5(a)-(c)) were created to simplify the overall representation and to readily isolate the SAN representing the intrinsic pathway. The presence of multiple feedback reactions and several enzymes, each activating multiple reactions, make the representation of the complete extrinsic pathway difficult to develop as a single system, particularly since model completion requires iterative model refinements. The composed model for coagulation incorporates the five SANs which represent the subsegments of the pathway constructed such that at least one common place exists for overlap of the joined SAN pairs. These common places are necessary to complete the composed model of coagulation and are presented in Table 1 601 in FIG. 6.

Model Refinement Against Experimental Observations

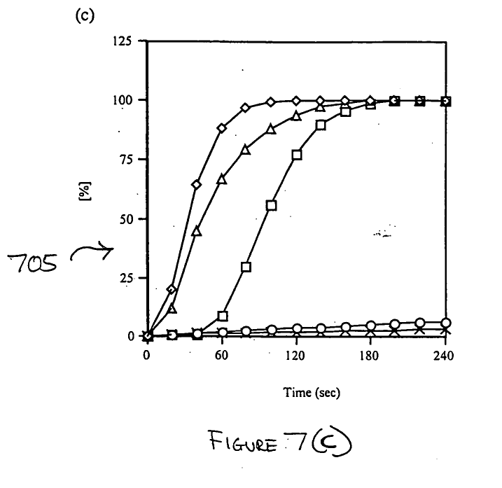

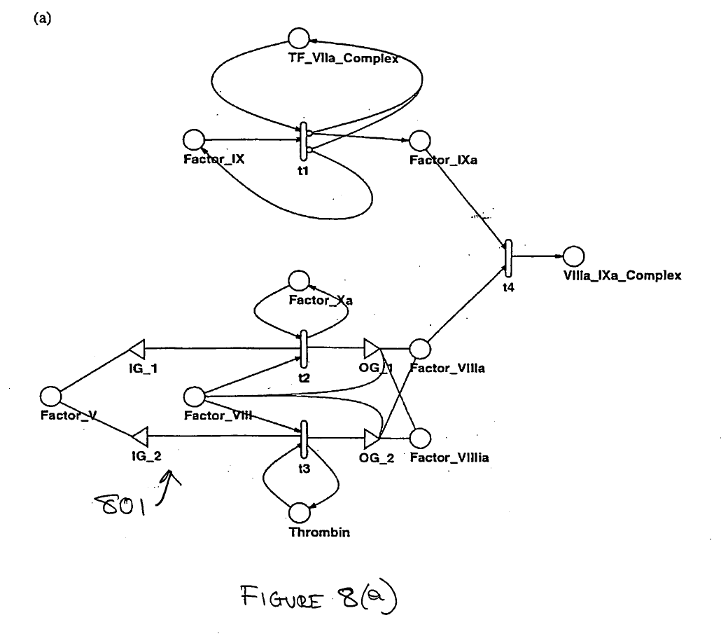

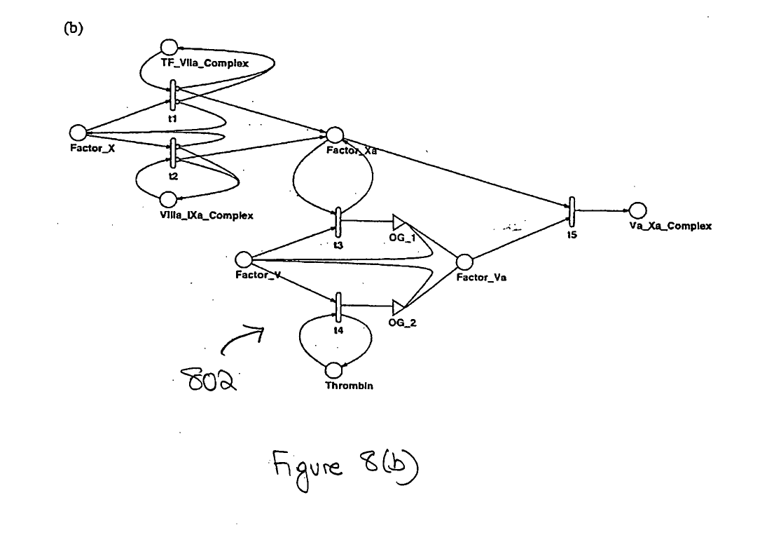

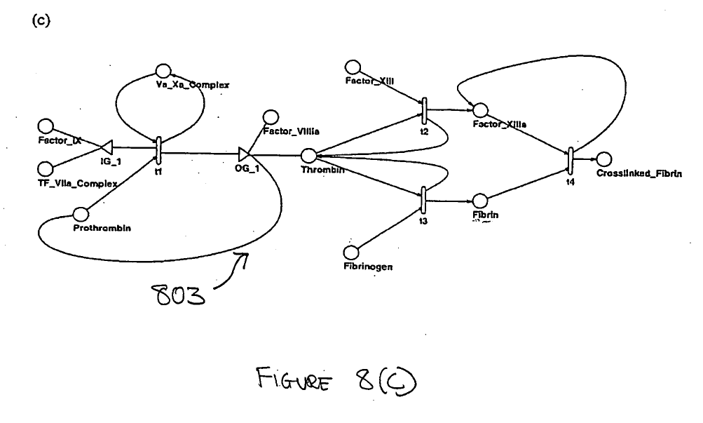

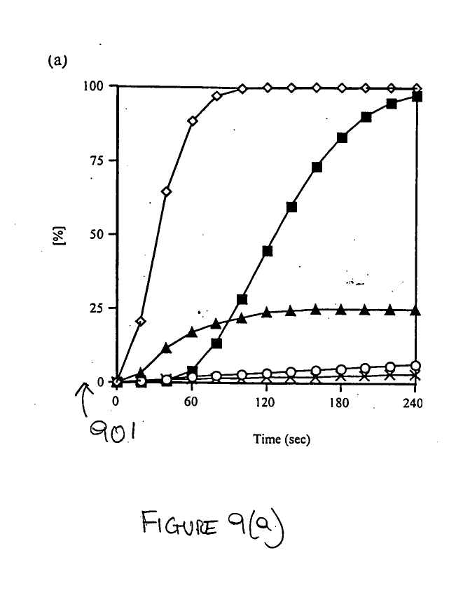

The process of creating a computer representation which enables simulation and modeling of the coagulation system in normal behavior and disease states requires access to experimental kinetic data. The initial model is iteratively refined and tested to reproduce the behavior of the dynamic, experimental data. The kinetic profile of the extrinsic pathway to thrombin was used to accomplish the refinement of the components and the composed model because this study provided a consistent set of data on substrate and product evolution during coagulation. This five-component system of blood coagulation (factor V, factor VIII, factor IX, factor X, and prothrombin) spanned the critical components of the extrinsic coagulation pathway. FIG. 7(a) presents the experimental data 701 for the activation reactions of the five factors listed above. Thrombin concentrations which reach greater than 100% represent the effects of the superactive intermediate meizothrombin which is not included in the present model.

The refinement of the initial model state to the accurate, final model of blood coagulation was an iterative process which focused on the reactions of the five components listed above. To demonstrate this process, we present the steps required to transform the initial. Va_Xa_Binding SAN (FIG. 5(c)) to one which is used in the final model. The initial SAN was based on the stoichiometric relationships of the proteins involved in the five reactions. Activity t1 represents the enzymatic activation reaction of factor X to factor Xa by the tissue factor-factor VIIa complex which is produced in the initiation SAN (FIG. 5(a)). Activity t2 represents the similar enzymatic activation produced by the factor VIIIa-factor IXa complex which is produced in the VIIIa_IXa_Binding SAN (FIG. 5(b)). Activities t3 and t4 represent the enzymatic activation of factor V to factor Va by factor Xa and thrombin which is produced in the Clot_Formation SAN (FIG. 5(d)), respectively. Finally, activity t5 shows the formation of the complex between factor Va and factor Xa which is used downstream in the Clot_Formation SAN. The two components whose responses are to be calibrated in this SAN are factor V and factor X. The graph 703 shown in FIG. 7(b) shows the response of the initial model for these factors in addition to the other factors of the extrinsic pathway plotted against simulation time. It is apparent that the initial model is inadequate as it does not produce the desired output upon simulation. The first component to be refined was factor X because it is upstream of factor V in the reaction pathway. If factor V had been refined first, it is likely that the subsequent refinement of factor X would not be independent of factor V and would necessitate additional modifications. From the experimental data, it was determined that factor X activation behaved linearly with respect to time in which only 2.5 percent of the available factor X was activated through the first 240 seconds. To reproduce these results, the initial model would have to be altered to produce a much slower rate of factor X activation since the initial model shows factor X being activated to forty percent through 240 seconds. This was accomplished by creating two cases for activity t1, and subsequently t2. Case 1 would result in the same 1:1 reaction of factor X activation to factor Xa. Case 2 would introduce a null reaction, i.e., the substrate would be placed back into the factor X pool and would not be converted to factor Xa. The introduction of the second case creates the ability to produce a factor X activation curve that could vary between the original initial model output (forty percent through 240 seconds) if Case 1 has a selection probability of 1.0, to an output of zero percent activation if Case 2 has a selection probability of 1.0. The case probabilities used to produce accurate simulation results were 0.002 (0.2 percent) for the activation reaction and 0.998 (99.8 percent) for the null reaction. These values produce the corresponding factor X activation curve 705 shown in FIG. 7(c).

The second component of the Va_Xa_Binding SAN is the factor V activation curve which results from activities t3 and t4. Based on the simulation results of the initial model, it is apparent that the slope of the activation curve of factor V must be increased to approximate the experimental data. The most direct solution is to increase the amount of tokens, i.e., the number of molecules, transferred when an activity is fired. This does not change the stoichiometry of the system, but it does provide a more rapid process for factor V activation. By increasing the amount of token flow through the activity to 100, i.e., all of the factor V is enzymatically activated to factor Va in one step, the model simulates results which are much closer to the experimental data. The activation curve in the model predicts activation at a later time than is observed experimentally. This is resolved by increasing the rate at which the activity is accessed in the model to attain a more accurate representation of the experimental kinetics. The simulation behavior of this resulting model is shown in FIG. 7(c).

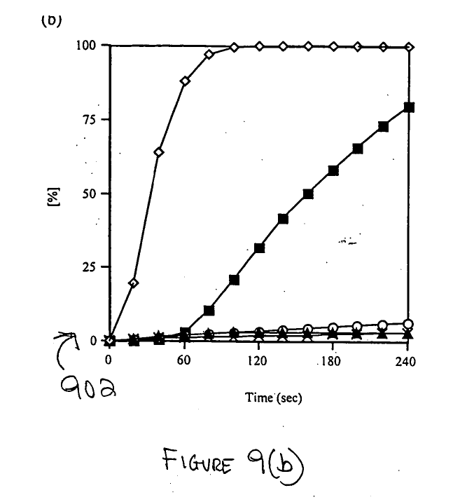

An analogous process of iterative refinement to the model was also performed on the factor VII and factor IX activation reactions represented in the VIIIa_IXa_Binding SAN (FIG. 5(b)) and the thrombin activation reaction represented in the Clot_Formation SAN (FIG. 5(d)). These resulted in an accurate model capable of reproducing the experimental results of Lawson, et al. The VIIIa_IXa_Binding SAN is affected in much the same way as was the Va_Xa_Binding SAN, as might be expected by the high degree of similarity between the architecture of the two SANs. The activity representing the activation of factor IX by the tissue factor-factor VIIa complex was altered to have two cases in order to produce accurate simulation results, one representing the activation reaction (Case 1 with a case probability of 0.75) and one representing a null reaction (Case 2 with a case probability of 0.25) where the substrate was not converted to product. The activation curve of factor VIII was produced exactly like the factor V activation curve. That is, the throughput through the activity was increased to 100 and the rate of activity access was increased three-fold. In order to produce the delay experimentally observed in factor VIII activation with respect to factor V activation, an additional criteria was established. Eighty percent of factor V was activated via thrombin or factor Xa, and then and only then would factor VIII activation be allowed to proceed at the specifications set. The method of calibration for the thrombin activation curve was similar to that of both factor V and factor VIII in that the number of tokens through the activity was increased to 100. The rate of activity access however was left at its initial value. The activation of prothrombin had to be delayed from its initial, immediate activation to one that more closely resembles the delay of fifty seconds observed from the experimental results. In this case, the key points were activation of factor IX and activation of factor VII, such that when one percent of factor IX was activated and five percent of factor VII was activated, thrombin activation would proceed. This produced the delay shown in the experimental findings. The iterative process evolved to the final model 801-803 shown in FIGS. 8(a)-(c), where only the SANs that were manipulated during the iterative process are presented. The final model is now considered to be a competent representation of the normal coagulation system.

The characteristics of the resulting model provide access to significant flexibility for further hypothesis evaluation. The incorporation of multiple SANs into one composed model allows the user to add and remove molecular species, reactions, pathways, etc. For example, to refine the model against the experimental data, it is easier to limit the analysis to only one or two SANs rather than the complete model to focus on a single reaction or group. In addition, parts of the system can be modeled or defined as appropriate. For example, the initiation SAN simplifies the representation of the actual process of coagulation initiation, i.e., the enzyme responsible for the activation of factor VII is not represented. The nature of the tool and the approach allows for the model to be developed with limited information and for inclusion of the missing segments to the representation even after the crude model has been initially refined. A system-wide refinement is a much more limited process than the refinement of the initial model.

Hemophilia Analysis

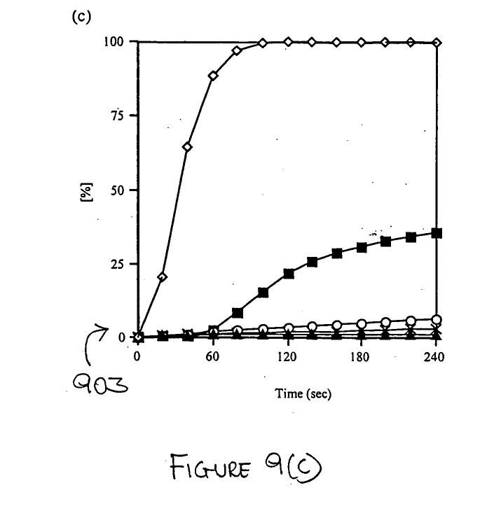

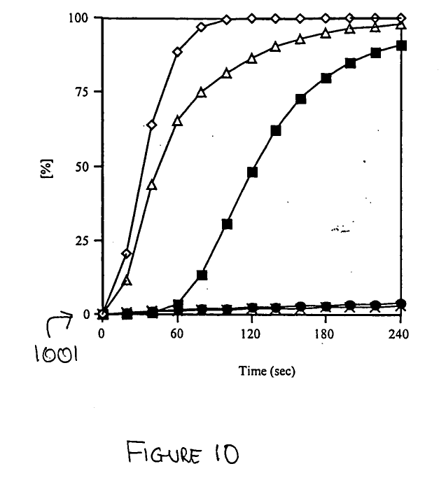

Completion of a competent model for normal blood coagulation enables its use to study various natural single site mutations to provide information on how-the system responds upon incorporation of these changes. The most prevalent inherited bleeding abnormalities of the coagulation system are hemophilia A and B, caused by X-linked, recessive, genetic defects in factors VIII and IX, respectively. The primary clinical observations in these diseases include prolonged or spontaneous bleeding, and depend on the severity of the respective disease, i.e., expression level of the defective gene. These two distinct genetic diseases provide a logical test to evaluate the sensitivity of our coagulation model for disease modeling since both involve perturbation to factors which are key components of our model. Hemophilia A simulations are presented in FIGS. 9(a)-(c) in which three graphs show the results of simulations of mild (25% factor VIII activity) 901, moderate (3% factor VIII activity) 902, and severe (1% factor VIII activity) 903 hemophilia A. The deviation from normal levels of factor VIII is due to mutation or non-expression and does not contribute to clot formation. The simulation under these separate conditions show that the varying degrees of severity impact coagulation by altering the resulting thrombin curve which is directly proportional to the rate of clot formation. As the severity of the disease increases, i.e., factor VIII decreases, the amount of thrombin generated decreases in amount formed, and an increasing lag in time required to achieve a specific level increases dramatically. Thus, mild hemophilia A only shifts the time course while moderate and severe also significantly limit total production.

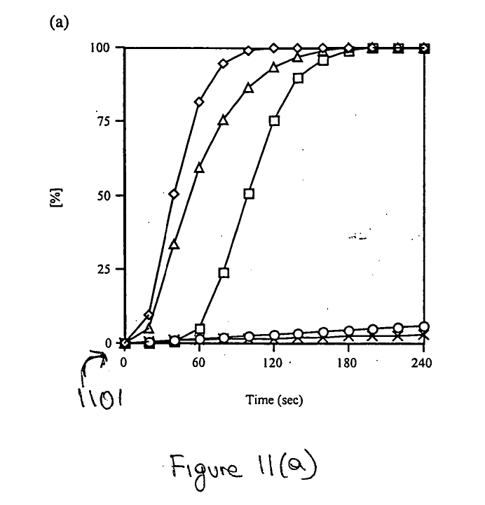

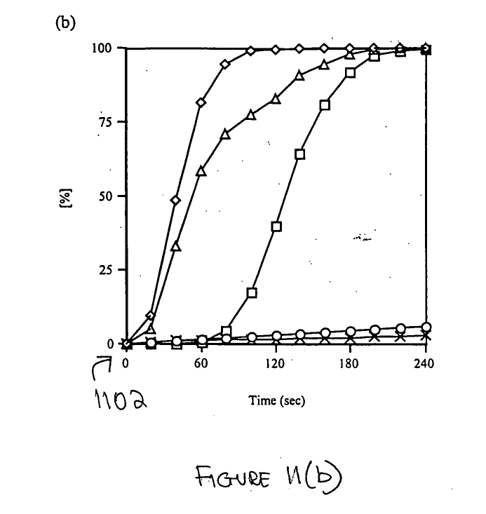

An analogous study was performed with the factor IX defect hemophilia B. The results 1001 of this study are presented in FIG. 10 and show that when the amount of useful factor IX is decreased by fifty percent, the thrombin activation curve shows a similar delay in clot formation to the results presented for mild hemophilia A, but also with a limiting thrombin production level.

Sensitivity Analysis of Coagulation Factors

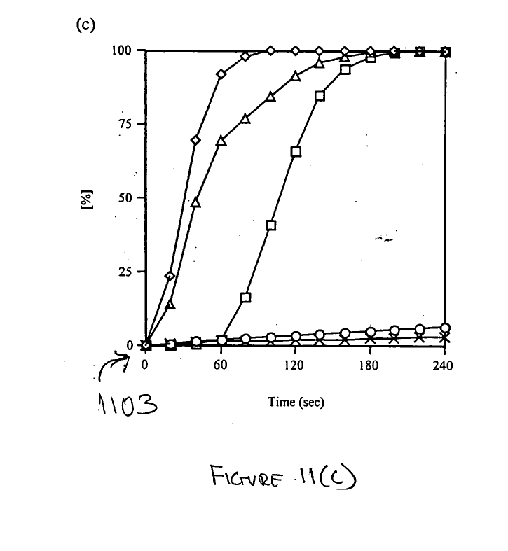

In addition to the analysis of hemophilia, above, we have also performed sensitivity analysis on various coagulation factors to identify critical points in the blood coagulation pathway. The goal of this analysis was to identify potential control sites for therapeutic intervention and disease analysis in non-hemophilia disorders. Factor VII was the first factor studied even though the factor VII activation curve was not refined directly against experimental data in the model. Sensitivity analysis of this component is still appropriate because of the overall competency of the model and because we are focusing on the downstream effects of factor VII perturbations. There are two reactions involved with factor VII in the initiation SAN presented in FIG. 5(a), the binding reaction with tissue factor and the activation reaction. To evaluate factor VII sensitivity, therefore, there are three potential situations to evaluate: 1) deficiency or defect of the binding reaction; 2) the activation reaction; or 3) both simultaneously. We have performed simulations of all three cases and present the results in FIGS. 8(a)-(c). FIG. 11 (a) shows the result 1101 on the extrinsic pathway if twenty-five percent of factor VII is defective for binding with tissue factor. That is, for every four molecules that normally bind to tissue factor, only three are able to effectively bind. FIG. 11(b) shows the result 1102 of what happens when factor VII is twenty-five percent defective towards activation. FIG. 11(c) shows the result 1103 what happens when factor VII is either twenty-five percent deficient or twenty-five percent defective towards both the binding and activation reactions. From this initial analysis, all of the deficiency experiments predict an increased thrombin time; thirty-three percent in FIG. 11(a), sixty-three percent in FIG. 11(b), and thirty-eight percent in FIG. 11(c).

In addition to disease modeling, a complete SAN representation of blood coagulation can be used to show the effects of therapeutics used to combat the various diseases affecting coagulation. For example, one of the most common treatments of hemophilia A is replacement therapy where normal or recombinant factor VII is introduced into the bloodstream to promote normal coagulation. This replacement can be modeled, and therefore the SAN model can show how much of the replaced factor is needed for normal coagulation levels by predicting the effectiveness of the therapy. In addition to replacement therapies, other therapies can also be modeled. Therapeutic drugs and their intended behaviors can be added to the representation such that their effectiveness or lack thereof can be shown. In addition to primary effects, this type of modeling allows the user to address other areas of interest such as dose dependency and side effects.

Other embodiments of the invention will be apparent to those skilled in the art from a consideration of the specification and the practice of the invention disclosed herein. It is intended that the specification be considered as exemplary only, with the true scope and spirit of the invention also being indicated-by the following claims.

What is claimed is:

1. A method of evaluating a biochemical pathways models using clinical data, comprising the steps of:
(a) representing the biological model within a computing system, in the form of a hierarchy of Petri nets or stochastic activity nets, wherein the nodes of the net represent biological or biochemical components, and the arcs correspond to the flow of biochemical components in the model;
(b) recording a time series of measurements of some of the biochemical components described by the model, wherein these measurements are made on samples from one or more humans or other living organisms;
(c) storing those observations as a data set in the computing system, which performs the subsequent steps as automated computations;
(d) for each time series in the stored data set, calculating the rate of change of some or all variables with respect to time, and augmenting the data set with that information;
(e) for each input to a network node, labeling the input with the expected effect of an increase in token flow at the input on each output of the node, either “increasing” or “decreasing”;
(f) comparing an observed pattern of increases and decreases of measured biochemical levels versus the expected pattern of increases and decreases in flow rates of tokens through the corresponding nodes; and
(h) marking the biochemical components for which the expected effect is an increase while the biochemical measurements show a decrease, or vice versa, wherein the marking indicates that the model does not describe the behavior of those biochemical components.

2. The method according to claim 1, wherein step (d) comprises selecting a subset of the data set based on patient demographic data or a history of prior treatment before calculating a rate of change of some or all variables from the selected subset of the data set.

3. The method according to claim 1, further comprising: for models that have no markings in step (h), identifying nodes whose outputs are connected to inputs of a number of nodes that is higher than the average connectivity of outputs of nodes to inputs of other nodes.

4. The method according to claim 3, wherein the identified nodes are identified as key regulatory factors.

5. A method of evaluating a biological model comprising the steps of:
(a) representing the biological model within a computing system, in the form of a hierarchy of Petri nets or stochastic activity nets, wherein the nodes of the net represent biological or biochemical components, and the arcs correspond to the flow of biochemical components in the model;
(b) recording a set of measurements of some of the biochemical components described by the model, wherein these measurements are made on samples from one or more humans or other living organism patients, wherein the set of patients is divided into subsets representing patients with a disease, various diseases, and/or healthy patients;
(c) storing the measurements a data set in the computing system, which performs the subsequent steps as automated computations;
(d) for subsets of the data, calculating the change of some or all measured variables with respect to the corresponding value of that variable for healthy patients or diseased patients in a different subsets, and augmenting the data set with that information;
(e) for each input to a network node, labeling the input with the expected effect of an increase in token flow at the input on each output of the node, either “increasing” or “decreasing”;
(f) comparing the pattern of increases and decreases of measured biochemical levels in various disease states versus the expected pattern of increases and decreases in flow rates of tokens through the corresponding nodes; and
(g) marking the biochemical components for which the expected effect is an increase while the biochemical measurements show a decrease, or vice versa, the marking indicating that the model does not adequately describe the behavior of those biochemical components.

6. The method according to claim 5, further comprising: (h) for models which are consistent in that they have no markings from step (g) above, further marking nodes whose outputs are connected, directly or indirectly, to inputs of a relatively large number of nodes compared to the average connectivity of the nodes in the network, the marking indicating that the node is a potential key regulatory factor.

7. The method according to claim 5, wherein step (d) comprises further restricting the subsets of the data set, based on patient demographic information or history of prior treatment;

8. The method according to claim 6, wherein the step (b) comprises recording measurements of a subset of patients comprising only diseased patients; and step (h) comprises using cluster analysis to stratify the disease into clusters.

9. The method according to claim 6, wherein the step (b) comprises recording measurements of both diseased and healthy patients, and step (h) comprises using the identified nodes for developing a biochemical test for the disease being studied.

10. The method according to claim 6, wherein the step (b) comprises recording measurement of patients in various stages of a disease, and step (h) comprises using the identified nodes to measure or predict disease progression stages.
